(19) United States
(12) Patent Application Publication
(10) Pub. No.: US 2002/0061282 A1
(43) Pub. Date: May 23, 2002

(54) Tooth whitening composition and method employing dicarboxylic acid whitening agent

(76) Inventor: Constantine Georgiades, East Brunswick, NJ (US)

Correspondence Address: Darryl C. Little, Attorney for Applicant, Warner Lambert Company, 201 Tabor Road, Morris Plains, NJ, 07950, US

(21) Appl. No.: 09/946,213

(22) Filed: Sep. 5, 2001

Related U.S. Application Data
(60) Provisional application No. 60/239,296, filed on Oct. 11, 2000.

(51) Int. Cl. A61K 7/16

(52) U.S. Cl. 424/49

(57) ABSTRACT

A tooth-whitening composition includes at least one dicarboxylic acid, such as oxalic acid malonic acid, tartaric acid, and/or a salt thereof as a whitening agent, and preferably contains the essential oils thymol; methyl salicylate; menthol; and eucalyptol. The composition can be provided in a variety of forms, including a mouthwash, a toothpaste, a tooth gel, a tooth powder, an oral film and a lozenge. The composition is effective for whitening teeth by removing extrinsic stains from external surfaces of the teeth. The composition also helps to maintain the teeth white by hindering the deposition of extrinsic stains on the external surfaces of teeth.

This application claims the benefit of U.S. Provisional Application No. 60/239,296, filed on Oct. 11, 2000, the entirety of which is hereby incorporated by reference as if fully set forth herein.

### SPECIFICATION

### FIELD OF THE INVENTION

This invention relates to tooth whitening compositions and more specifically to such compositions employing oxalic acid as a tooth whitening agent.

### BACKGROUND OF THE INVENTION

The majority of people consider clean, white teeth to be aesthetically very desirable. Teeth with extrinsic stains are objectionable to the general public both on the basis of cosmetic appearance and also socially as an indication of poor oral hygiene. Because the acquired pellicle, which coats the teeth, has a natural tendency to stain, most people will form some unsightly extrinsic stains on their teeth over time. This staining process is promoted by (1) the ingestion of tannin-containing foods and beverages such as coffee, tea, or red wine; (2) the use of tobacco products; and (3) exposure to certain cationic substances such as tin, iron, and chlorhexidine.

Currently, the most widely practiced method for the control of extrinsic stains is daily tooth brushing with dentifrices. However, tooth brushing alone is not capable of completely preventing stain formation. Areas of the dentition, which are commonly missed during tooth brushing, such as the interproximal tooth surfaces and the lingual areas of the anterior teeth, are very prone to stain accumulation. Once the stain has formed, it is very difficult to remove without obtaining a professional dental cleaning.

Dental bleaching is one technique proposed to remove extrinsic stains from the teeth. According to U.S. Pat. No. 5,989,526 to Aaslyng et al., one of the most commonly used bleaching techniques employed by dentists combines the use 30% hydrogen peroxide with heat and light treatment to speed up the oxidation reaction (i.e., the removal of stains). An alternative bleaching method disclosed by Aaslyng et al. comprises bathing the teeth in a 10% urea peroxide (carbamide peroxide) composition contained in a mouthpiece fitted to the teeth. Aaslyng et al. purports to improve upon prior art bleaching methods by providing dental bleaching compositions comprising at least one oxidoreductase, such as a laccase, an oxidase and/or a peroxidase. These compositions are said to be safer than compositions employing other bleaching agents, such as hydrogen peroxide.

Many other dental bleaching agents have been proposed, including oxalic acid and its salts. For example, Chapple reported in 1877 that a discolored tooth could be restored to its normal translucency by placing one to two drops of a saturated solution of oxalic acid in pure water into the pulp-chamber of the tooth for about three to six minutes, and thoroughly washing the solution from the tooth with warm water alone prior to filling the tooth. Of course, this method was limited to professional use and was targeted to intrinsic rather than extrinsic dental stains. Moreover, Chapple's inspiration for his report was an earlier article of Bogue (14(1) Dental Cosmos 1 (1872)) which explained that the technique was designed to bleach the internal staining caused by extravasation of blood into the dentine.

WO 98/23219 (Sibner) discloses a topical dental bleaching composition for use with a laser, wherein the composition comprises a bleaching agent, an inert gelling agent, a plurality of discrete laser enhancing particles and a pH modifier. Although the bleaching agent can be oxalic acid, hydrogen peroxide at a concentration of about 5% to about 70% by volume is preferred. The laser-enhanced bleaching method of Sibner is obviously intended for professional use only.

WO 92/09261 (Andelbeek) discloses an in vitro method for cleaning dentures, wherein the dentures are treated in a first bath containing sodium hypochlorite and soda, and a second bath containing oxalic acid. The first bath is intended to bleach the dentures and the oxalic acid of the second bath is intended to remove tartar from the dentures.

Oxalic acid and salts thereof have also been employed as desensitizing agents in oral care compositions. For example, U.S. Pat. No. 4,057,621 to Pashley et al. discloses a method of desensitizing hypersensitive dentin and cementum by applying to the dentin and cementum a desensitizing amount of a composition which has as the essential ingredient a member selected from the group consisting of a mono- and di-substituted alkali metal and ammonium oxalate in aqueous solution. The essential ingredient is applied in a desensitizing amount in a concentration of between about 2.0% by weight of said ingredient and a weight percent which is solution saturation. The alkali metal or ammonium oxalate used as the active treating agent penetrates into the tubules and fibriles of the dental dentin layer to reduce or eliminate fluid movement within the tubules or fibriles, thus rendering the dentin incapable of transducing normally painful stimuli to the pulp in the form of fluid movement.

U.S. Pat. No. 5,766,328 to Nakabayashi et al. discloses a dental composition for relieving dentin hypersensitivity comprising (A) an aqueous emulsion component (1) which contains polymer particles as emulsion particles having a diameter smaller than that of a dentinal tubule and forming an agglomerate larger than the diameter of a dentinal tubule when they react with a calcium compound and (2) which has a metal ion concentration in a dispersing medium of 1,000 ppm or less, and (B) a water-soluble organic acid component or a water-soluble salt component thereof, a calcium salt of the organic acid being insoluble or hardly soluble in water. Component B can be oxalic acid.

U.S. Pat. No. 5,849,267 to Collins et al. discloses a desensitizing anti-tartar dentifrice comprising a dentifrice vehicle including an alkali metal carboxymethyl cellulose gelling agent, an alkali metal polyphosphate or a phosphono antitartar agent, and a tooth pain inhibiting potassium salt. In certain less preferred embodiments, the potassium salt is potassium oxalate. The potassium salt is provided in a desensitizing amount, which is generally about 2 to 10 wt. %. of the dentifrice.

WO 92/04006 (Neirinckx) discloses oral desensitizing compositions comprising a source of a first ionic species and a source of a second ionic species, wherein the two ionic species combine to form a precipitate that reduces or eliminates fluid movement within the tubules or fibriles. The ionic species can be, e.g., calcium oxalate or strontium oxalate.

Other patent documents disclosing the use of oxalic acid and salts thereof as desensitizing agents in oral care compositions include, e.g., U.S. Pat. No. 5,352,439 to Norfleet et al., U.S. Pat. No. 5,505,933 to Norfleet et al., U.S. Pat. No. 5,906,809 to Hack et al.

Oxalic acid and salts thereof have also been employed as anti-caries agents in oral care compositions. For example, U.S. Patent No. 5,026,539 to Jackson et al. discloses an oral hygiene composition comprising: (a) up to 10% by weight of a source of hydrogen citrate ions; (b) up to 7% by weight of an oxalate salt; and (c) an orally acceptable excipient, wherein the composition has a pH of from 4 to 7. The composition is said to have anti-caries activity.

EP 0 242 977 (Poile et al.) discloses oral hygiene compositions comprising up to 7% by weight of the composition of alkali metal oxalate or alkaline earth metal oxalate, 20 to 1500 ppm of fluoride ions, and a dentally acceptable excipient. The composition has a pH of 4 to 1 0 and excludes mouthwash compositions comprising solubilized aluminum compounds. The oxalate is preferably present in a concentration of 0.0025 to 7 wt. %. The combination of oxalate and fluoride ions is said to provide an enhanced anticaries effect. No bleaching effect is disclosed.

U.S. Pat. No. 4,591,384 to Akahane et al. discloses the use of oxalic acid as a reducing agent in dental cement compositions. The compositions comprise a metal oxide, a tannic acid derivative that is sparingly soluble in water and a water-soluble reducing agent. The reducing agent can be oxalic acid. The compositions are said to suffer from little, if any, discoloration, and have improved setting properties and reduced solubility. The reducing agent is provided in an amount of 0.005 to 5 wt. % based on the total weight of the composition.

Despite the foregoing developments in the oral care art, it is desired to provide oral care compositions comprising at least one dicarboxylic acid for treating extrinsic dental stains. It is further desired to provide such compositions in the form of a mouthwash, toothpaste, film, pastille, nougat, micro-capsule, tooth gel, and the like, that can be routinely and effectively employed by consumers as a part of their daily hygiene regimen.

All references cited herein are incorporated herein by reference in their entireties.

### SUMMARY OF THE INVENTION

Accordingly, the invention provides a composition for oral administration, said composition comprising a whitening agent selected from the group consisting of dicarboxylic acids and salts of dicarboxylic acids, and the essential oils thymol, methyl salicylate, menthol and eucalyptol.

Also provided is a composition for oral administration, said composition consisting essentially of: a whitening agent selected from the group consisting of dicarboxylic acids and salts of dicarboxylic acids; thymol; methyl salicylate; menthol; eucalyptol; a dentally acceptable vehicle selected from the group consisting of water, ethanol, 1-propanol and mixtures thereof; a buffering agent; a non-ionic surfactant; and a sweetener.

The invention also provides a method for whitening teeth, said method comprising applying a composition of the invention to external surfaces of teeth to remove extrinsic stains from said external surfaces.

In addition, an oral care kit is provided. The kit comprises: an oral care composition comprising a whitening agent selected from the group consisting of dicarboxylic acids and salts of dicarboxylic acids; and a container containing said oral care composition, wherein said container is labeled with indicia indicating that said oral care composition whitens teeth.

### BRIEF DESCRIPTION OF THE DRAWINGS

The invention will be described in conjunction with the following drawings in which like reference numerals designate like elements and wherein:

FIGS. 1, 2 and 3 are histograms showing the tooth whitening effects of several embodiments of the invention contrasted with the tooth whitening effects of other compositions; and

FIG. 4 is a histogram showing the stain prevention effects of several embodiments of the invention contrasted with the stain prevention effects of other compositions.

### DETAILED DESCRIPTION OF PREFERRED EMBODIMENTS

Particularly preferred compositions of the invention combine the surprisingly effective tooth whitening benefits of dicarboxylic acids and salts of dicarboxylic acids with the antimicrobial and other benefits of the LISTERINE® line of oral care products (available from the Warner-Lambert Consumer Group of Pfizer). Thus, preferred compositions of the invention comprise at least one dicarboxylic acid (and/or at least one salt of a dicarboxylic acid) and the essential oils (thymol, methyl salicylate, menthol and eucalyptol) thought to be largely responsible for the benefits common to the various members of the LISTERINE® line of oral care products.

Particularly preferred dicarboxylic acids for use as whitening agents in accordance with the invention include oxalic acid, malonic acid, tartaric acid and salts thereof. Oxalic acid and its salts are most preferred. Suitable dicarboxylic acid salts include, but are not limited to, sodium, potassium, zinc, iron, calcium, magnesium, and copper salts of, e.g., oxalic acid, malonic acid and tartaric acid.

In certain embodiments, the whitening agent can be a tricarboxylic acid, such as citric acid or a salt thereof.

The whitening agent is present in compositions of the invention at a tooth-whitening effective concentration. A tooth-whitening effective concentration is preferably less than 10% (or about 10%), more preferably less than 5% (or about 5%), and most preferably from 0.05% (or about 0.05%) to 1% (or about 1%). Percentages referred to herein are in weight-volume (w/v) units for liquid embodiments and weight-weight (w/w) units for semi-solid and solid embodiments.

Preferred compositions according to the present invention also include essential oils. Essential oils are volatile aromatic oils that are synthetic or are derived from plants by distillation, expression or extraction. Essential oils usually carry the odor or flavor of the plant from which they are obtained. If used in the dentifrice compositions of this invention, essential oils provide anti-gingivitis activity. Some of these essential oils also act as flavoring agents. The essential oils of this invention include, but are not limited to, thymol, menthol, methyl salicylate (wintergreen oil) and eucalyptol.

Thymol, also known by the chemical formula 5-methyl 2-(1-methylethyl) phenol, is obtained from the essential oil of Thymus vulgaris Labiatae and Monarda punctata Labiatae. Thymol is a white crystalline powder with an aromatic odor and taste. Thymol is soluble in organic solvents but only slightly soluble in deionized water.

Menthol is isolated principally from the oil of Mentha arvensis. In its commercial form, menthol is available as L-menthol crystals obtained from a process involving cooling of the oil. Fractional distillation of peppermint oil that usually contains from about 40% to about 65% menthol represents another important source of menthol. Synthetic sources of L-menthol are also available.

Eucalyptol is derived from the eucalyptus tree. Having a camphoraceous odor and cooling taste, this essential oil is often combined with other essential oils such as menthol in confection formulations to impart medicinal effect. Combinations of menthol and eucalyptol are widely used. Particularly preferred uses of the menthol-eucalyptol combination include, according to the present invention, dentifrices such as toothpastes or dental gels.

Methyl salicylate is the main ingredient in many essential oils, constituting about 99% of oil of wintergreen (Gaultheria procumbens) and sweet birch (Betula lenta). Methyl salicylate, which has a distinctive refreshing aroma, is used widely in mouthwashes, chewing gums and other oral and pharmaceutical preparations.

The amounts of essential oils that can be used in the compositions of the present invention are from 0.001 (or about 0.001) to 1% (or about 1%) thymol, 0.001 (or about 0.001) to 1% (or about 1%) methyl salicylate, 0.001 (or about 0.001) to 15% (or about 15%) menthol and 0.001 (or about 0.001) to 1% (or about 1%) eucalyptol, wherein said amounts are clinically effective in inhibiting gingivitis. More preferably, a composition according to the present invention contains about 0.064% thymol, about 0.060% methyl salicylate, about 0.042% menthol and about 0.092% eucalyptol, wherein said amounts are clinically effective in inhibiting gingivitis.

Compositions of the invention preferably include ingredients additional to oxalic acid, salts of oxalic acid and the essential oils. Such additional ingredients include, e.g., antimicrobial agents, fluorine-providing compounds, anti-tartar compounds, anticalculus agents, acidifiers, abrasives, surfactants, buffering agents, binders, thickeners, humectants, sweeteners, desensitizing agents, flavorants, colorants, and preservatives. The ingredients are combined in a hydrous or anhydrous vehicle to form a solid (e.g., a toothpowder, compressed tablet, or lozenge ), a semi-solid (e.g., a paste, gel, micro-capsule, nougat, or pastille), or a liquid (e.g., a mouthwash).

The compositions of this invention can be substantially solid or pasty in character such as dental cream, toothpaste, toothpowder, lozenges, films, micro-capsule, compressed tablet, pastille, nougat or chewing gum. Solid or pasty oral compositions contain polishing materials. Typical polishing materials are abrasive particulate materials having particle sizes of up to about 20 microns. Non-limiting illustrative examples include water-insoluble sodium metaphosphate, potassium metaphosphate, tricalcium phosphate, dihydrated calcium phosphate, anhydrous dicalcium phosphate, dicalcium phosphate, calcium pyrophosphate, magnesium orthophosphate, trimagnesium phosphate, calcium carbonate, alumina, aluminum silicate, zirconium silicates, silica, bentonite, and mixtures thereof. Polishing materials are generally present in an amount from about 1% to about 99% by weight of the composition. Preferably, it is present in amounts from about 20% to about 75% in toothpaste, and from about 70% to about 99% in toothpowder. An example of a preferred toothpaste of the invention is shown in Table 1, below.

TABLE 1

| Ingredient | Percent W/W |
|---|---|
| Glycerin USP Special | 6.000 |
| Xanthan Gum K6B166 | 1.0000 |
| Sodium Carboxymethyl | 0.6000 |
| Cellulose Type 12M31 | |
| Sodium Carboxymethyl | 0.6000 |
| Cellulose USP Type | |
| Water Deionized | 33.6413 |
| Sorbitol Solution USP | 32.0000 |
| Sodium Monofluorophosphate | 0.7600 |
| (100%) | |
| Saccharin Sodium | 1.0000 |
| (Spray Dried, FCC) | |
| Sodium Phosphate | 0.2500 |
| (Monobasic) Anhydrous | |
| Sodium Phosphate | 0.0300 |
| (Dibasic) Anhydrous | |
| Acid Benzoic USP | 0.1500 |
| Titanium Dioxide USP | 0.3500 |
| Hydrated Silicon Dioxide | 7.0000 |
| Silica Amorphous, Synthetic | 11.0000 |
| (Sylodent 750) | |
| Sodium Lauryl Sulfate- | 1.5000 |
| Washed & Dried | |
| Water (Deionized) | 2.5000 |
| Thymol NF | 0.3000 |
| Methyl Salicylate NF | 0.0600 |
| Menthol USP | 0.1750 |
| Eucalyptol | 0.1000 |
| Peppermint Oil | 0.1369 |
| (Farwest Terpeneless VR) | |
| Flavor (Koolmist Toothpaste | 0.1175 |
| Flavor No. BK-16762-88B) | |
| FD and C Blue No. 1 | 0.0010 |
| D and C Yellow No. 10 | 0.0001 |
| Oxalic Acid | 0.4125 |
| Total | 100.0000 |

In clear gels, it is preferred to provide a polishing agent of colloidal silica and alkali metal aluminosilicate complexes since they have refractive indices close to the refractive indices of gelling agent liquid systems commonly used in dentifrices.

In preferred embodiments, the oral composition is a liquid such as a mouthwash or rinse (hereinafter collectively referred to as a “mouthwash”). The total amount of the liquid vehicle in a mouthwash composition is typically in the range of about 70% to about 99.9% by weight of the composition. The pH value of such mouthwash compositions is preferably from about 2.0 to about 8.5, preferably from 3 (or about 3.0) to 7.5 (or about 7.5), more preferably from 3.0 (or about 3.0) to below 7 (or about 7), and most preferably about 4.2. A pH below 2 would be irritating to the oral cavity. A pH greater than 8.5 would result in an unpleasant mouth feel.

The pH of the composition is adjusted using suitable food or pharmaceutical grade bases or acids. Suitable bases include, e.g., sodium hydroxide, and the like. Suitable acids include, e.g., phosphoric acid, benzoic acid, citric acid, or other tricarboxylic acids, and the like. The exact amount of base or acid added depends on the final pH and buffer capacity desired.

The pH of the composition is preferably buffered. Common buffer systems, in addition to the oxalate buffering system described in this invention, include phosphoric acid and sodium phosphate salts, or citric acid and sodium citrate. Suitable buffers for use in this invention include citric acid-sodium citrate, phosphoric acid-sodium phosphate, sodium monobasic phosphate, sodium dibasic phosphate, acetic acid-sodium acetate, succinic acid-sodium succinate, aconitic acid-sodium aconitate and benzoic acid-sodium benzoate in amounts up to about 1%, preferably from about 0.05% to about 0.75% of the composition, and most preferably from about 0.1% to about 0.5% of the composition.

Preferably, the vehicle for liquid forms is a water-alcohol mixture, wherein the ratio of water to alcohol is in the range of from about 1:1 to about 20: 1, preferably about 3:1 to about 20:1 and most preferably about 3:1 to about 10:1 by volume. The most preferred mouthwash or mouth rinse compositions comprise from 0 to about 30% by volume (v/v%) alcohol, such as ethanol.

Oral liquid compositions can also contain surface active agents in amounts up to about 5%. Surface active agents are organic materials, which afford complete dispersion of the composition throughout the oral cavity. The organic surface active material can be non-ionic, amphoteric, or cationic (with cationic being preferred).

Non-ionic surface active agents include condensates of sorbitan mono-oleate with from 20 to 60 moles of ethylene oxide (e.g., “Tweens” a trademark of ICI United States, Inc.), condensates of ethylene oxide with propylene oxide and condensates of propylene glycol (“Pluronics” a trademark of BASF-Wyandotte Corp.).

Other suitable non-ionic surfactants useful in the present invention include polyoxyethylene castor oil derivatives which are ethoxylated hydrogenated castor oils. These surfactants are prepared by hydrogenating castor oil and treating the hydrogenated product with from about 10 to about 200 moles of ethylene glycol. These ethoxylated hydrogenated castor oils are known by the non-proprietary name of polyethylene glycol (PEG) hydrogenated castor oils, in accordance with the Dictionary of the Cosmetics, Toiletries and Fragrance Association, 3rd Edition, which name is used in conjunction with a numeric suffix to designate the degree of ethoxylation of the hydrogenated castor oil product, i.e., the number of moles of ethylene oxide added to the hydrogenated castor oil product. Suitable PEG hydrogenated castor oils include PEG 16, 20, 25, 30, 40, 50, 60, 80, 100 and 200. A preferred PEG hydrogenated castor oil surfactant is Cremophor RH 60, a commercially available product from BASF-Wyandotte, Parsippany, New Jersey.

Other suitable non-ionic surfactants are the condensation products of an alpha-olefin oxide containing 10 to 20 carbon atoms, a polyhydric alcohol containing 2 to 10 carbons and 2 to 6 hydroxyl groups and either ethylene oxide or a mixture of ethylene oxide and propylene oxide. The resultant surfactants are heteric polymers having a molecular weight in the range of about 400 to about 1600 and containing 40% to 80% by weight of ethylene oxide, with a alpha-olefin oxide to polyhydric alcohol mole ratio in the range of about 1:1 to 1:3.

Amphoteric surfactants useful in the present invention include zwitterions having the capacity to act as either an acid or a base. They are generally non-irritating and non-staining. Non-limiting examples of suitable amphoteric surfactants include cocoamidopropyldimethylsultaine and cocodimethylbetaine (commercially available from Lonza Chem. Co. under the trade-names Lonzaine CS and Lonzaine 12C, respectively).

Cationic surface active agents suitable for use in the invention include, e.g., quaternary ammonium compounds, CPC, chlorhexidine, alexidine, and hexetidine. Such cationic surfactants can enhance the antimicrobial activity of the oral care composition of the invention.

Other suitable antimicrobial agents include, e.g., non-cationic antimicrobial agents, such as phenolic and bisphenolic compounds, halogenated diphenyl ethers, benzoate esters and carbanilides.

Illustrative of the phenolic antimicrobial compounds, which include the halogenated salicylanilides, are 2-phenylphenol, 4-chlorophenol, 4-chloro-2-methylphenol, 4-chloro-3-methylphenol, 4-chloro-3,5-dimethylphenol, 2,4-dichloro-3,5-dimethylphenol, 3,4,5,6-tetrabromo-2-methylphenol, 5-methyl-2-pentylphenol, 4-isopropyl-3-methylphenol, 5-chloro-2-hydroxydiphenylmethane, 4′,5-dibromosalicylanilide, 3,4′,5-trichlorosalicylanilide, 3,4′,5-tribromosalicylanilide, 2,3,3′,5-tetrachlorosalicylanilide, 3,3′,4,5′-tetrachlorosalicylanilide, 3,5-dibromo-3′-trifluoromethylsalicylanilide and 5-n-octanoyl-3′-trifluoromethylsalicylanilide.

Suitable bisphenolic compounds include 2,2′-methylenebis(3,4,6-trichlorophenol), 2,2′-methylenebis(4-chlorophenol), 2,2′-methylenebis(4-chloro-6-bromophenol), bis(2-hydroxy-3,5-dichlorophenyl) sulphide and bis(2-hydroxy-5-chlorophenyl) sulphide. In embodiments, these antibacterial agents can be employed in the form of their zinc derivatives, many of which are disclosed in U.S. Pat. No. 4,022,880.

Exemplifying the class of the halogenated hydroxydiphenyl ethers are the compounds 2′,4,4′-trichloro-2-hydroxy-diphenyl ether and 2,2′-dihydroxy-5,5′-dibromo-diphenyl ether.

Another well-known class of non-cationic antimicrobial agents are the esters of p-hydroxybenzoic acid, especially the methyl, ethyl, propyl, isopropyl, butyl, isobutyl, hexyl, heptyl and benzyl esters.

Halogenated carbanilides can also be used in embodiments, which class is typified by 3,4,4′-trichlorocarbanilide, 3-trifluoromethyl-4,4′-dichlorocarbanilide and 3,3′,4-trichlorocarbanilide.

Other known substantially water-insoluble non-cationic antimicrobial agents can also be used, for example 2,4-dichlorobenzyl alcohol, 3,4-dichlorobenzyl alcohol and 3-(4-chlorophenoxy)-propan-1,2-diol.

The above-mentioned antimicrobial agents that are suitable for use in dentifrices are not antibiotics. Antibiotics are preferably avoided so as to avoid the risk of resistant strains of bacteria developing.

The antimicrobial agent will usually be used in an amount of 0.01 to 5%, preferably 0.05 to 1% by weight of the dentifrice. A mixture of antimicrobial agents may, of course, be used.

Fluoride-releasing compounds are preferably used in the compositions of the present invention. These compounds may be fully or slightly water soluble, release fluoride ions or fluoride-containing ions in water and do not react with other components in the composition. It is well known that compositions containing fluoride-releasing compounds help prevent dental caries. Typical fluoride-releasing compounds are inorganic fluoride salts such as water-soluble alkaline earth metal, alkali metal, and heavy metal salts. Sodium monofluorophosphate, sodium fluoride, stannous fluoride and mixtures of these compositions are preferred.

The amount of fluoride-releasing compound present in the compositions of this invention must be nontoxic. The specific amount depends upon the type of fluoride-releasing compound employed, the solubility of the fluoride-releasing compound and the formulation of the composition. In general, the fluoride-releasing compound will be present in an amount by weight of up to about 1.2%, preferably from about 0.1% to about 1.0%, and most preferably from about 0.175% to about 0.8% of the composition so as to provide 75-1500 ppm fluoride ion.

The compositions of the present invention preferably contains at least one anticalculus agent in an amount ranging from about 0.25% to about 10%, preferably from 1.5% to 7%. Suitable anticalculus agents include, e.g., polyphosphates and pyrophosphates, such as disodium pyrophosphate, dipotassium pyrophosphate, tetrasodium pyrophosphate and tetrapotassiumpyrophosphate, and mixtures thereof. Zinc salts are also suitable anticalculus agents.

The compositions of the present invention may additionally contain sweeteners, flavorants and colorants.

In the instance where auxiliary sweeteners are utilized, the present invention contemplates the inclusion of those sweeteners well known in the art, including both natural and artificial sweeteners. Thus, additional sweeteners may be chosen from the following non-limiting list:

A. Water-soluble sweetening agents, such as monosaccharides, disaccharides and polysaccharides, such as xylose, ribose, glucose, mannose, galactose, fructose, dextrose, sucrose, maltose, partially hydrolyzed starch, or corn syrup solids and sugar alcohols such as sorbitol, xylitol, mannitol and mixtures thereof.

B. Water-soluble artificial sweeteners, such as Sucralose®, the soluble saccharin salts, i.e., sodium, or calcium saccharin salts, cyclamate salts, acesulfame-K and the like, and the free acid form of saccharin.

C. Dipeptide based sweeteners such as L-phenylalanine methyl ester and materials described in U.S. Pat. No. 3,492,131 and the like.

In general, the amount of sweetener will vary with the desired amount of sweetness selected for a particular composition. This amount will normally be 0.01% to about 40% by weight. The water-soluble sweeteners described in category A above, are preferably used in amounts of about 5% to about 40% by weight, and most preferably from about 10% to about 20% by weight of the final composition. In contrast, the artificial sweeteners described in categories B and C are preferably used in amounts of about 0.005% to about 5.0% and most preferably about 0.05% to about 2.5% by weight of the final composition. These amounts are ordinarily necessary to achieve a desired level of sweetness independent from the flavor level achieved from flavorants.

Suitable flavorants include, e.g., both natural and artificial flavors, such as mints (e.g., peppermint spearmint, etc.), citrus flavors such as orange and lemon, artificial vanilla, cinnamon, various fruit flavors and the like. Both individual and mixed flavors are contemplated. The flavorings are generally utilized in amounts that will vary depending upon the individual flavor, and can, for example, range in amounts of about 0.1% to about 6% by weight of the final composition.

The colorants useful in the present invention include pigments which can be incorporated in amounts of up to about 2% by weight of the composition. Also, the colorants can include other dyes suitable for food, drug and cosmetic applications (i.e., FD&C dyes) and the like. The materials acceptable for the foregoing spectrum of use are preferably water-soluble. Illustrative examples include the indigo dye known as FD&C Blue No. 2, which is the disodium salt of 5,5-indigotindisulfonic acid, FD&C Green No. 1, which is a triphenylmethane dye and is the monosodium salt of 4-[4-N-ethyl-p-sulfobenzyl amino)diphenyl-methylene]-[1-(N-ethyl-N-p-sulfoniumbenzyl)-2,5-cyclohexadie nimine]. A full recitation of all FD&C and D&C colorants useful in the present invention and their corresponding chemical structures can be found in the Kirk-Othmer Encyclopedia of Chemical Technology, 3rd Edition, in Volume 6, at pages 561-595.

The present invention also involves a method for whitening teeth, comprising applying to the surface of the teeth the compositions of this invention as described earlier. The compositions can be applied to the teeth and gums by any conventional means, such as brushing, spraying, painting or rinsing of the oral cavity and the like. Compositions of the invention are effective for whitening teeth by removing extrinsic stains from external surfaces of the teeth. The compositions also help to maintain the teeth white by hindering the deposition of extrinsic stains on the external surfaces of teeth.

The invention will be illustrated in more detail with reference to the following Examples, but it should be understood that the present invention is not deemed to be limited thereto.

### EXAMPLES

### Examples 1 to 22

The stain removal method used in this investigation was a modification and improvement of that described by Stookey et al., “In-vitro removal of stain with dentifrices.” 61(11) J. Dent. Res. 1236-89 (Nov. 1982), which is incorporated herein by reference.

Approximately 4 mm squares of dental enamel were cut from bovine permanent incisors, using a diamond-cutting disk. Using a mold, the enamel squares were embedded in clear polyester casting resin (obtained from NATCOL Crafts, Inc., Redlands, Calif. 92373) to provide 1.5 cm square blocks with the labial surface exposed. The top surface of the polyester blocks was ground flush with the leveled labial surface of the enamel squares by means of a dental model trimmer. The surface was smoothed by hand sanding on 400-grit emery paper using water as the lubricant until all grinding marks were removed. Finally, the top surface of the blocks was hand-polished to a mirror finish using a water slurry of GK1 072 calcined Kaolin (median particle size of 1.2 microns) on a cotton cloth. The finished specimens were examined under a dissecting microscope and rejected for testing if surface imperfections were observed.

In preparation for the formation of artificial stained pellicle on the enamel, the specimens were etched for 60 seconds in 0.2M HCl, followed by a 30-second immersion in a saturated solution of sodium carbonate. A final etch was performed with 1% phytic acid for 60 seconds, after which the specimens were rinsed with deionized water and attached to the staining apparatus.

The staining apparatus was constructed to provide alternate immersion into the staining broth and air-drying of the specimens. The apparatus consisted of an aluminum platform base which supported a Teflon rod (¾-inch in diameter) connected to an electric motor, which by means of a speed reduction box, rotated the rod at a constant rate of 1.5 rpm. Threaded screw holes were spaced at regular intervals along the length of the rod. The tooth specimens were attached to a rod by first gluing the head of a plastic screw to the back of the specimen, then screwing the tooth onto the rod. Beneath the rod was a removable, 300-mL capacity trough, which held the staining broth.

The staining broth was prepared by adding 1.02 g of instant coffee, 1.02 g of instant tea, and 0.75 g of porcine gastric mucin (obtained from Nutritional Biochemicals Corporation, Cleveland, Ohio 44128) to 250 mL of sterilized trypticase soy broth. Approximately 50 mL of a 24-hour Micrococcus luteus culture was also added to the stain broth. The apparatus, with the enamel specimens attached and the staining broth in the trough, was then placed in an incubator at 37 □C. with the specimens rotating continuously through the staining broth and air. The staining broth was replaced once every 24 hours for ten consecutive days. With each broth change, the trough and specimens were rinsed and brushed with deionized water to remove any loose deposits. On the eleventh day, the staining broth was modified by the addition of 0.03 g of FeCl₃□6H₂O, and this was continued with daily broth changed until the stained pellicle film on the specimens was sufficiently dark (L* score range of 32 to 35). Then, the specimens were removed from the staining broth, brushed thoroughly with deionized water, placed in a humidor, and refrigerated until used.

Procedure for Sixty-minute Whitening Assay of Examples 1 to 22

Stained enamel specimens mounted in clear resin are removed from a refrigerated humidor, rinsed gently with distilled water and blotted with a Kimwipe. The specimens are then air-dried at room temperature for one hour. Three specimens are used for each treatment solution tested.

The intensity of the extrinsic stained pellicle on each specimen is measured with a Minolta Chroma Meter (Minolta CR-321 Chroma Meter with 450 circumferential illumination/0° viewing angle and 3 mm aperture; available from Minolta Corporation, 101 Williams Drive, Ramsey, N.J., 07446). The specimen is centered under the detector of the Chroma Meter to enhance reproducability of results. An average of 3 absorbency readings using the L*a*b* scale are taken for each measurement prior to treatment with the test solutions. These components are analyzed separately to determine the specific changes in the whiteness (L*), red-green color range (a*), and yellow-blue color range (b*).

The specimens are placed into 50 mL plastic beakers containing 20 mL of test solution for 60 minutes. The beakers are covered with foil (to minimize evaporation and spillage) and are swirled using an empty Gyrotary Water Bath Shaker model G76 at speed setting 6. After 60 minutes, the specimens are removed from the beakers and rinsed gently with distilled water. The specimens are then air-dried for one hour before measuring the intensity of the stain post-treatment with test solutions. This reading provides the L*a*b* values that are used to calculate the amount of stain removed by a test solution.

A series of treatment solutions were tested for whitening effect using the assay described above. Example 1 tested the whitening effect of water only. Example 2 tested the whitening effect of a commercial lot of COOLMINT LISTERINE® (a product of the Warner-Lambert Consumer Group of Pfizer) without additional additives. The solutions used in Examples 3 and 8-22 were prepared by adding various potential whitening agents to a commercial lot of COOLMINT LISTERINE®. The solutions used in Examples 4-7 had the formulations shown in Table 2, wherein the ingredients were added to a commercial lot of COOLMINT LISTERINE® to make 1 liter of each solution. The pH of the solutions were adjusted to 4.2 using 6 N NaOH.

TABLE 2

| | Amount (in grams) | | | |
|---|---|---|---|---|
| Ingredient | Example 4 | Example 5 | Example 6 | Example 7 |
| Menthol | 0.323 | 0.323 | 0.323 | 0.325 |
| Thymol | 0.638 | 0.637 | 0.638 | 0.642 |
| Methyl Salicylate | 0.690 | 0.661 | 0.670 | 0.668 |
| Eucalyptol | 0.925 | 0.92 | 0.916 | 0.923 |
| Pluronic | 2.503 | 2.498 | 2.497 | 2.504 |
| Na Saccharin | 1.169 | 1.17 | 1.172 | 1.172 |
| Oxalic Acid | 0.498 | 1.006 | 2.505 | 5.036 |
| Alcohol | 183.60 | 183.49 | 183.51 | 184 |
| Sorbitol | 199.97 | 199.94 | 200.24 | 200.04 |
| Propanol | 5.02 | 5.001 | 4.976 | 5.004 |
| Flavor | 0.86 | 0.855 | 0.85 | 0.859 |
| Color | 0.50 | 0.51 | 0.52 | 0.513 |

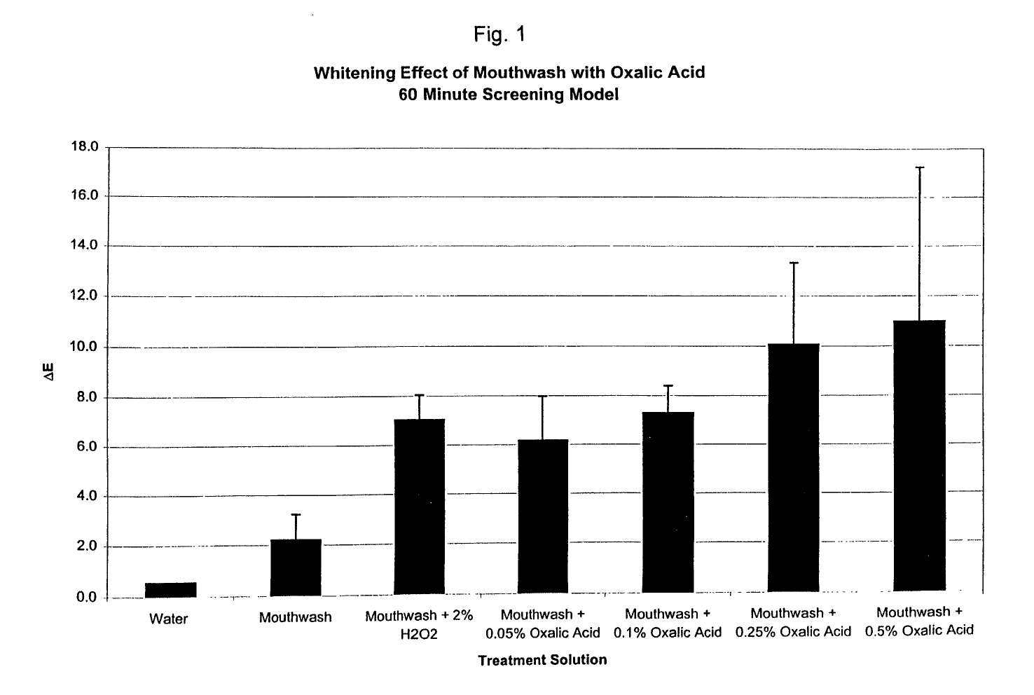

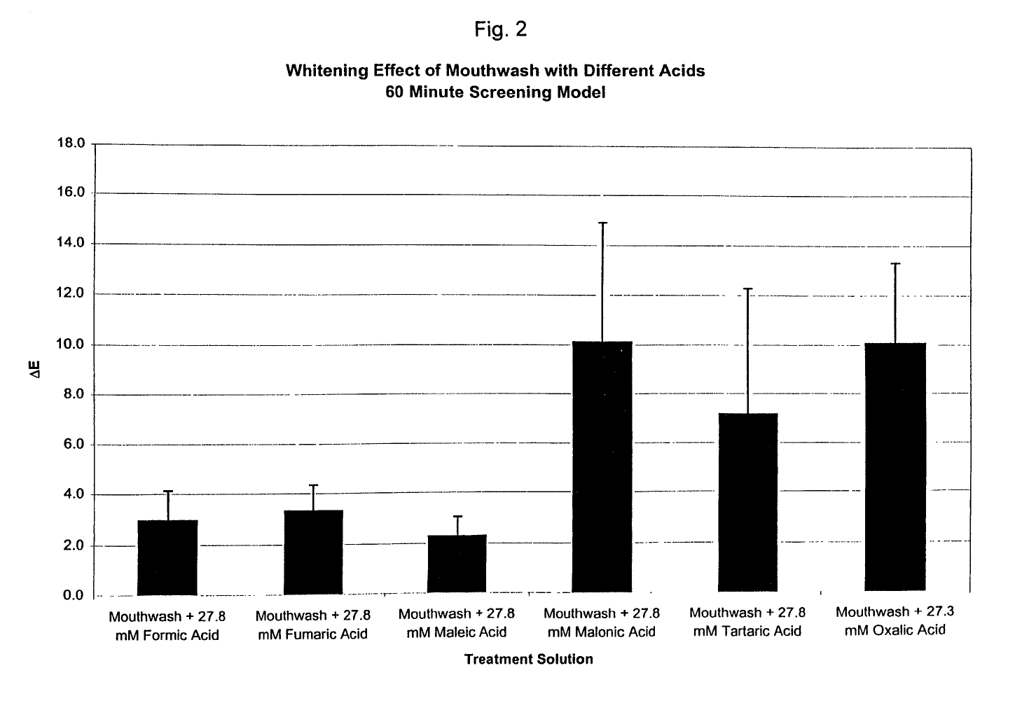

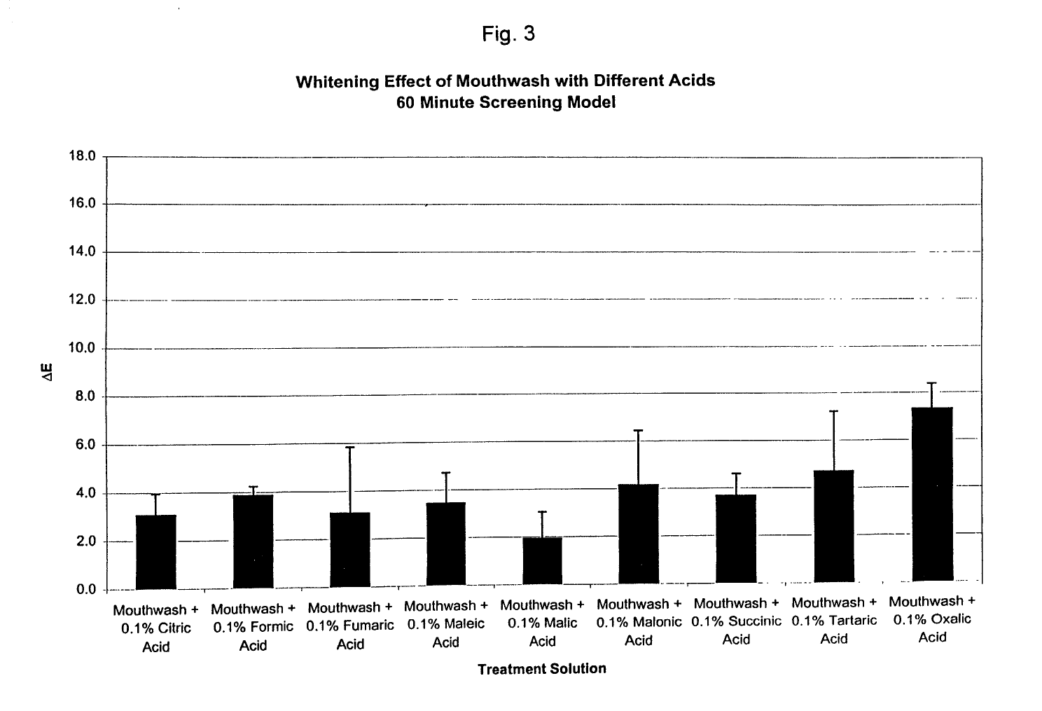

As shown by the results tabulated in Table 3 and illustrated in FIGS. 1-3, compositions of the invention (containing oxalic acid or salts thereof) outperformed compositions containing other potential whitening agents, including a mouthwash composition containing hydrogen peroxide at a higher concentration than that of oxalic acid as demonstrated by relative ΔE values below. ΔE is a measure of the overall change in extrinsic stain (ΔE) (see definition infra).

The difference between the pre-test and post-test readings for each color factor (L*, a*, and b*) represents the ability of the test solutions to remove extrinsic stained pellicle from teeth over time. The primary outcome variable used to assess efficacy is the overall change in extrinsic stain (ΔE), and is calculated using the CIELAB equation:

ΔE=[(ΔL*)²+(Δa*)²+(Δb*)²]^{½}.

Where: ΔL*, Δa*, and Δb* were the stain removal scores for the individual components of the L*a*b* scale. The tooth whitening compositions of the present invention preferably provide a ΔE measure value of greater than 2, preferably greater than 3 and most preferably greater than 4.

TABLE 3

| | Treatment Solution | | Standard |
|---|---|---|---|
| Example | (all % are w/v) | ΔE | Deviation |
| 1* | Water | 0.54 | |
| 2* | Mouthwash | 2.2 | 1.03 |
| 3* | Mouthwash + 2% H₂O₂ | 7.05 | 0.99 |
| 4 | Mouthwash + 0.05% Oxalic Acid | 6.16 | 1.81 |
| 5 | Mouthwash + 0.1% Oxalic Acid | 7.30 | 1.09 |
| 6 | Mouthwash + 0.25% Oxalic Acid | 10.06 | 3.25 |
| 7 | Mouthwash + 0.5% Oxalic Acid | 10.99 | 6.25 |
| 8 | Mouthwash + 27.8 mM Formic Acid | 2.96 | 1.17 |
| 9 | Mouthwash + 27.8 mM Fumaric Acid | 3.30 | 1.03 |
| 10 | Mouthwash + 27.8 mM Maleic Acid | 2.28 | 0.75 |
| 11 | Mouthwash + 27.8 mM Malonic Acid | 10.12 | 4.79 |
| 12 | Mouthwash + 27.8 mM Tartaric Acid | 7.16 | 5.11 |
| 13 | Mouthwash + 27.3 mM Oxalic Acid | 10.06 | 3.25 |
| 14 | Mouthwash + 0.1% Citric Acid | 3.05 | 0.90 |
| 15* | Mouthwash + 0.1% Formic Acid | 3.86 | 0.38 |
| 16 | Mouthwash + 0.1% Fumaric Acid | 3.07 | 2.76 |
| 17 | Mouthwash + 0.1% Maleic Acid | 3.44 | 1.29 |
| 18 | Mouthwash + 0.1% Malic Acid | 1.92 | 1.11 |
| 19 | Mouthwash + 0.1% Malonic Acid | 4.14 | 2.3 |
| 20 | Mouthwash + 0.1% Succinic Acid | 3.67 | 0.94 |
| 21 | Mouthwash + 0.1% Tartaric Acid | 4.7 | 2.51 |
| 22 | Mouthwash + 0.1% Oxalic Acid | 7.30 | 1.09 |

*Comparative examples.

### Examples 23 to 31

Procedure for Stain Prevention Assay of Examples 23 to 31

The staining method used in this investigation was a modification and improvement at described by Addy et al., 2 Journal of Dentistry 95-99 (1995).

Staining Solution

A standard tea solution was prepared fresh every day. Twenty grams of a commercially available brand of tea (Lipton “Brisk” tea, either loose or removed from tea bags) was added to 2 L of freshly boiling USP water and allowed to boil for 2 minutes with gentle stirring. The flask was then removed from the hot plate and placed into a water bath of cold tap water for 10 minutes. The water in the bath was then replaced with fresh cold tap water and the flask remained for an additional 25 minutes. The flask was then removed from the water bath and allowed to sit for another 10 minutes on the counter. The tea solution was gravity filtered through 3 gauze pads (12 ply, 4×4 inch, USP type VII gauze). The filtered tea solution was then vacuum filtered with a Buchner Funnel through Whatman #1 filter paper.

Labware Setup

Test tube racks were set up with forty 25×150 mm glass test tubes in an array having 10 columns and 4 rows. Each test tube in the first row contained 25 mL of test solution. The test tubes in the second and fourth rows each contained 25 mL of USP water. The test tubes in the third row contained 25 mL of tea solution.

The substrate used to mimic teeth was opaque polymethylmethacrylate (PMMA) tiles. They measured 1 in.×⅝ in. and had a hole drilled in the middle of the top of the tile. Lengths of CROMEL A® wire (Hoskins Manufacturing Co., Detroit, Mich.) were cut and bent into hooks. The straight end of each wire was placed into a #4 black rubber stopper and the hooked-end of each wire was threaded through a tile hole. This arrangement allowed the tile to hang in the center of the test tube without touching the sides.

Stain Measurements

The intensity of the extrinsically formed stain on the PMMA tiles was measured by taking diffuse reflectance absorbency readings with a Minolta Chroma Meter (Minolta CR-321 Chroma Meter with 45° circumferential illumination/0° viewing angle and 3 mm aperture; available from Minolta Corporation, 101 Williams Drive, Ramsey, N.J., 07446). The light source was the CIE standard illuminant C, and measurements were recorded in the L*a*b* color space. The meter was calibrated with White Calibration Plate CR-A45 (No. 21833028) before measurements were taken each day. Each measurement was taken with the Multi Measure option on, giving an average of three readings as the result. The top of each tile was aligned with the inside of the instrument base plate to assure measurements were being taken at the same spot each time. Baseline readings of each individual PMMA tile were taken before the start of treatment. Treated tiles were allowed to air dry at room temperature for at least one hour before measurements were made, in order to obtain a consistent reading.

Treatment

The PMMA tiles were hung from the wire hooks attached to rubber stoppers. The tiles were then immersed in the test solution, dipping up and down 10-15 times to remove air bubbles, for 2 minutes (Row 1 of the array). The tiles were then rinsed with water for approximately 5 seconds by dipping quickly up and down in USP water (Row 2 of the array). Next the tiles were immersed in the tea solution, dipping up and down 10-15 times to remove air bubbles, for 30 minutes (Row 3 of the array). Lastly the tiles were rinsed again in USP water for approximately 5 seconds by dipping quickly up and down (Row 4 of the array). This process is preferably repeated for a total of eight treatments per tile. The tiles were dried prior to measuring the stain formation. Stain measurements were taken after the eight treatments.

A series of treatment solutions were tested for stain prevention effect using the assay described above. Example 23 tested the stain prevention effect of water only. Example 24 tested the stain prevention effect of a commercial lot of COOLMINT LISTERINE® without additional additives. The solutions used in Examples 25-30 were prepared by adding various potential stain prevention agents to a commercial lot of COOLMINT LISTERINE®. The solution used in Example 31 was the same solution as prepared for Example 5 above. The pH of the solutions were adjusted to 4.2 using 6 N NaOH.

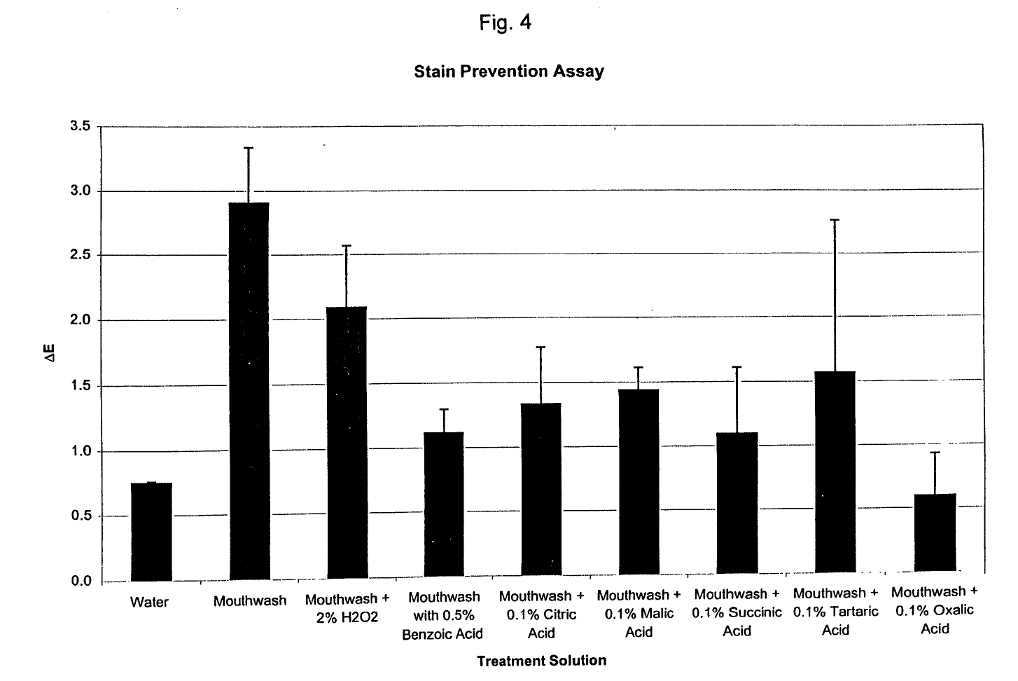

As shown by the results tabulated in Table 4 and illustrated in FIG. 4, compositions of the invention (containing a dicarboxylic acid or a salt thereof) outperformed compositions containing other potential stain prevention agents, including a mouthwash composition containing hydrogen peroxide at a higher concentration than that of oxalic acid.

TABLE 4

| | Treatment Solution | | Standard |
|---|---|---|---|
| Example | (all % are w/v) | ΔE | Deviation |
| 23* | Water | 0.75 | 0.01 |
| 24* | Mouthwash | 2.91 | 0.43 |
| 25* | Mouthwash + 2% H₂O₂ | 2.09 | 0.48 |
| 26* | Mouthwash + 0.5% Benzoic Acid | 1.11 | 0.18 |
| 27 | Mouthwash + 0.1% Citric Acid | 1.33 | 0.44 |
| 28 | Mouthwash + 0.1% Malic Acid | 1.44 | 0.17 |
| 29 | Mouthwash + 0.1% Succinic Acid | 1.09 | 0.52 |
| 30 | Mouthwash + 0.1% Tartaric Acid | 1.57 | 1.19 |
| 31 | Mouthwash + 0.1% Oxalic Acid | 0.60 | 0.33 |

*Comparative examples.

While the invention has been described in detail and with reference to specific examples thereof, it will be apparent to one skilled in the art that various changes and modifications can be made therein without departing from the spirit and scope thereof.

What is claimed is:

1. A composition for oral administration, said composition comprising:
a.) a whitening agent selected from the group consisting of dicarboxylic acids and salts of dicarboxylic acids; and
b.) an essential oil selected from the group consisting of thymol, methyl salicylate, menthol, eucalyptol and mixtures thereof
wherein the composition provides a stain removing AE of greater than about 2.2.

2. The composition of claim 1, wherein said whitening agent is a member selected from the group consisting of oxalic acid, malonic acid, tartaric acid and salts thereof.

3. The composition of claim 1, wherein said whitening agent is oxalic acid or a salt of oxalic acid.

4. The composition of claim 2, wherein said whitening agent is present in said composition at a tooth-whitening effective concentration.

5. The composition of claim 4, wherein said tooth-whitening effective concentration is less than about 10%.

6. The composition of claim 5, wherein said tooth-whitening effective concentration is less than about 5%.

7. The composition of claim 5, wherein said tooth-whitening effective concentration is about 0.05% to about 1%.

8. The composition of claim 5, wherein said composition has a pH of about 4.2.

9. The composition of claim 5, wherein a thymol concentration is about 0.001% to about 1%, a methyl salicylate concentration is about 0.001% to about 1%, a menthol concentration is about 0.001% to about 15% and a eucalyptol concentration is about 0.001% to about 1%.

10. The composition of claim 9, wherein said tooth-whitening effective concentration is about 0.05% to about 1%.

11. The composition of claim 10, wherein said composition has a pH of about 4.2.

12. The composition of claim 11, wherein said whitening agent is oxalic acid or a salt of oxalic acid.

13. A composition for oral administration, said composition consisting essentially of:
a.) a whitening agent selected from the group consisting of dicarboxylic acids andsalts of dicarboxylic acids;
b.) an essential oil selected from the group consisting of thymol, methyl salicylate, menthol, eucalyptol and mixtures thereof;
c.) a dentally acceptable vehicle selected from the group consisting of water, ethanol, 1-propanol and mixtures thereof,
d.) a buffering agent;
e.) a non-ionic surfactant; and
f.) a sweetener
wherein the composition provides a stain removing ΔE of greater than about 2.2.

14. The composition of claim 13, wherein said whitening agent is a member selected from the group consisting of oxalic acid, malonic acid, tartaric acid and salts thereof.

15. The composition of claim 14, wherein a thymol concentration is about 0.001% to about 1%, a methyl salicylate concentration is about 0.001% to about 1%, a menthol concentration is about 0.001% to about 15% and a eucalyptol concentration is about 0.001% to about 1%.

16. The composition of claim 15, wherein a whitening agent concentration is about 0.05% to about 1%.

17. The composition of claim 16, wherein said buffering agent comprises benzoic acid and a salt thereof.

18. The composition of claim 17, wherein said non-ionic surfactant comprises condensates of ethylene oxide with propylene oxide and condensates of propylene glycol.

19. The composition of claim 18, wherein said composition has a pH of about 4.2.

20. The composition of claim 19, wherein said whitening agent is oxalic acid or a salt of oxalic acid.

21. A method for whitening teeth, said method comprising applying the composition of claim 1 to external surfaces of teeth to remove extrinsic stains from said external surfaces without irradiating said teeth with whitening-agent enhancing radiation.

22. The method of claim 21, wherein said composition is a liquid and said applying comprises rinsing said composition over said external surfaces.

23. The method of claim 21, wherein said composition is a paste or a gel and said applying comprises brushing said composition on said external surfaces.

24. An oral care kit comprising:
a.) an oral care composition comprising a whitening agent selected from the group consisting of oxalic acid, malonic acid, tartaric acid and salts thereof wherein the composition provides a stain removing AE of greater than about 2.2; and
b.) a container containing said oral care composition, wherein said container is labeled with indicia indicating that said oral care composition whitens teeth.

25. The oral care kit of claim 24, wherein said indicia further indicates that said whitening agent is at least one of oxalic acid, malonic acid, tartaric acid and salts thereof. A 26. A composition for oral administration, said composition comprising:
a.) a whitening agent selected from the group consisting of citric acid and salts of citric acid; and
b.) an essential oil selected from the group consisting of thymol, methyl salicylate, menthol, eucalyptol and mixtures thereof
wherein the composition provides a stain removing ΔE of greater than about 2.2.
